(19) United States
(12) Patent Application Publication
(10) Pub. No.: US 2026/0233185 A1
(43) Pub. Date: Aug. 13, 2026

(54) METHOD FOR CONTROLLING A HEAT-CONSUMING HYDROCARBON CONVERSION PROCESS

(71) Applicant: BASE SE, Ludwigshafen am Rhein (DE)

(72) Inventors: Daniel KECK, Ludwigshafen am Rhein (DE); Rombout KELDERMANS, Antwerpen (BE); Pieter REYNIERS, Ludwigshafen am Rhein (DE); David VAN CAUWENBERGE, Antwerpen (BE); Joerg UNGER, Ludwigshafen am Rhein (DE); Stefan DAHMEN, Ludwigshafen am Rhein (DE)

(21) Appl. No.: 19/151,854

(22) PCT Filed: Jan. 30, 2024
(86) PCT No.: PCT/EP2024/052231

§ 371 Date: Jul. 29, 2025

(30) Foreign Application Priority Data
Jan. 31, 2023 (EP) 23154161.6

(51) Int. Cl. B01J 19/00; B01D 53/047; B01J 4/00; B01J 6/00; B01J 19/24; C01B 3/24; C01B 32/05; C07C 4/04

(52) U.S. Cl. CPC B01J 19/0013; B01J 4/008; B01J 6/008; B01J 19/245; C01B 3/24; C01B 32/05; C07C 4/04; B01D 53/047; B01D 2256/245; B01D 2257/108; B01D 2257/7025; B01J 2204/002; B01J 2219/00072; B01J 2219/00117; B01J 2219/00164; C01B 2203/0266; C01B 2203/1241

(57) ABSTRACT

The present invention relates to a method for controlling a process carried out in an integrated production plant, wherein the process comprises subjecting at least one hydrocarbon feed stream to hydrocarbon conversion in a heat-consuming hydrocarbon conversion unit, and wherein the process is controlled so that heat which cannot be supplied to the heat-consuming hydrocarbon conversion unit via one or more plant-external sustainable heat sources is suitably supplied by hydrogen which is obtained from a light hydrocarbon conversion off-gas stream obtained from the heat-consuming hydrocarbon conversion unit.

The present invention relates to a method for controlling a process carried out in an integrated production plant and further relates to said process as such, wherein said process comprises subjecting at least one hydrocarbon feed stream to hydrocarbon conversion in a heat-consuming hydrocarbon conversion unit. Further, the present invention relates to an integrated production plant in which said process and said method for controlling said process are carried out.

Usually, many basic hydrocarbon conversion processes are endothermic processes which require heat for being carried out. Examples therefor are processes carried out in plants which comprise crackers such as steam crackers, ammonia plants, or steam reformers. If such processes are carried out along the lines of a responsible ecologically-driven process management, the necessary heat will be provided making use of one or more plant-external sustainable heat sources such as solar energy, wind energy, hydro energy, tidal energy, geothermal energy, and biomass energy, ammonia obtained from renewable sources, biomethane, bio-LNG and H₂ obtained from renewable sources. However, the availability of such plant-external sustainable heat sources may vary over time, and for certain periods of time, the respective amount of heat may not be sufficient to meet the needs of the process. Certainly, for such cases, it may be generally conceivable to use, in addition to the plant-external sustainable heat sources, other plant-external non-sustainable heat sources, i.e. fossil heat sources such as coal, oil or gas. However, according to the above-mentioned ecologically-driven process management, this is not an entirely preferred procedure. Alternatively, according to said scenario, the hydrocarbon conversion process could be shut down and started up once the necessary amount of heat via the plant-external sustainable heat sources is sufficiently high again; it is needless to say that this alternative is not desirable per se from an economical point of view.

WO 2022/200256 A1 relates to systems and methods for producing olefins from hydrocarbons in electrically-heated cracking furnaces with reduced CO₂ emission.

EP 4 029 924 A1 relates to an integrated cracker and reactor system and related processes for carrying out chemical reactions, the system comprising at least one pyrolysis reactor and at least one hydrocarbon cracker unit.

WO 2021/205011 A1 relates to an ethylene plant and to a process for producing ethylene in such plant.

Therefore, it was an object of the present invention to provide a method for controlling a process which is carried out in an integrated production plant and which comprises subjecting at least one hydrocarbon feed stream to hydrocarbon conversion in a heat-consuming hydrocarbon conversion unit in such a way that in case the heat supply to said hydrocarbon conversion cannot be provided by plant-external sustainable heat sources, the least possible amount of plant-external fossil heat sources needs to be used.

Thus, the present invention relates to a method for controlling a process carried out in an integrated production plant, wherein the integrated production plant comprises
(1) supply means M_{F} for providing one or more hydrocarbon feed streams S_{F} to a unit U_{C} for heat-consuming hydrocarbon conversion to obtain from U_{C} one or more product streams S and a light hydrocarbon conversion off-gas stream S_{O} comprising CH₄;
(2) the unit U_{C} which comprises heating means M_{H} for providing heat to the hydrocarbon conversion, wherein said heating means M_{H} comprise means for generating heat from one or more heat sources H_{S} and wherein said one or more heat sources H_{S} comprise H₂;
(3) controllable supply means M_{E} for providing one or more plant-external sustainable heat sources H_{EXT-S}, one or more plant-external fossil heat sources H_{EXT-F}, and one or more plant-internal heat sources H_{INT}, said plant-internal heat sources H_{INT} comprising H₂, for passing at least one of said heat sources H_{EXT-S}, H_{EXT-F} and H_{INT} to the heating means M_{H};
(4) a methane pyrolysis unit U_{M} for subjecting the light hydrocarbon conversion off-gas stream S_{O} to methane pyrolysis to obtain a hydrogen-rich gas stream S_{H} and a carbon stream S_{K};
wherein the process comprises
(i) passing one or more heat sources according to (3) from M_{E} to M_{H} in an amount sufficient for generating heat in M_{H} meeting the heat demand E_{UC} of the hydrocarbon conversion in U_{C};
(ii) passing at least one hydrocarbon feed stream S_{F} into U_{C} and subjecting the at least one feed stream S_{F} to hydrocarbon conversion in U_{C}, obtaining the one or more product streams S and S_{O};
(iii) passing S_{O} into U_{M} and subjecting S_{O} to methane pyrolysis in U_{M}, obtaining S_{H} and S_{K};
wherein the method for controlling the process comprises
(a) determining the heat demand E_{UC} of the hydrocarbon conversion in U_{C} to be provided by M_{H};
(b) determining the amount E_{EXT-S} of heat available for U_{C} from the one or more plant-external sustainable heat sources H_{EXT-S};
(c) determining ΔE=E_{UC}−E_{EXT-S} and

| (c.1) | if ΔE > 0, | |
|---|---|---|
| | (c.1.1) | determining H_{2N}, the amount of H₂ necessary to produce ΔE in M_{H}; |
| | (c.1.2) | determining H_{2P}, the maximum amount of H₂, producible in U_{M} to be |
| | | contained in S_{H}, to produce an amount E_{H2P} of heat in M_{H}; |
| | (c.1.3) | determining ΔH = H_{2N} − H_{2P}; and |

| | |
|---|---|
| (c.1.3.1) | if ΔH > 0, |
| | controlling the supply means M_{E} so that H_{2P} is passed into M_{H} |
| | via S_{H}; |
| | wherein the process further comprises |

| | |
|---|---|
| (iv) | passing the stream S_{H} obtained from U_{M} through M_{E}, |
| | obtaining a stream S′_{H} being y volume-% of S_{H}, with y = |
| | 100; |
| (v) | passing the stream S′_{H} obtained from M_{E} as H_{INT} into M_{H}; |
| (vi) | passing at least one of the one or more plant-external |
| | fossil heat sources H_{EXT-F} via M_{E} into M_{H} in an amount |
| | sufficient to generate heat in an amount (ΔE − E_{H2P}) in M_{H}; |

| | |
|---|---|
| (c.1.3.2) | if ΔH ≤ 0, |
| | controlling the supply means M_{E} so that an amount of H₂ is |
| | passed into M_{H} via S_{H} which is sufficient to produce ΔE in M_{H}; |
| | wherein the process further comprises |

| | |
|---|---|
| (iv′) | passing the stream S_{H} obtained from U_{M} through M_{E}, |
| | obtaining a stream S′_{H} being y volume-% of S_{H}, with 0 < |
| | y ≤ 100; |
| (v′) | passing the stream S′_{H} obtained from M_{E} as H_{INT} into M_{H}; |
| (vi′) | passing the stream S′_{H} obtained from M_{E} as H_{INT} into M_{H}; |

| (c.2) | if ΔE ≤ 0, | |
|---|---|---|
| | controlling the supply means M_{E} so that no H₂ is passed into M_{H} via S_{H}; | |
| | wherein the process further comprises | |
| | (iv″) | passing the stream S_{H} through M_{E}, obtaining a stream S″_{H} being 100 |
| | | volume-% of S_{H}; |
| | (v″) | removing the stream S″_{H} from M_{E}. |

### Unit U_{C}

Generally, there are no specific restrictions which unit U_{C} for heat-consuming hydrocarbon conversion according to (1) is comprised in the plant with the proviso that from U_{C}, one or more product streams S and a light hydrocarbon conversion off-gas stream S_{O} comprising CH₄ are obtained. Preferably, the unit U_{C} for heat-consuming hydrocarbon conversion comprises a cracking unit U_{CC}.

Cracking is a petrochemical process wherein saturated hydrocarbons having long molecular structures are broken down, i.e. cracked, into smaller saturated or unsaturated molecules. Generally, crackers aim at producing light alkenes as valuable products, especially ethylene and propylene. Cracking processes include fluid catalytic cracking (FCC) and steam cracking.

Conventional steam cracking utilizes a pyrolysis furnace which has two main sections: a convection section and a radiant section. The hydrocarbon feedstock typically enters the convection section of the furnace as a liquid, or, in a case where light feedstocks are used, as a vapor, wherein it is typically heated and, if necessary, vaporized by indirect contact with hot off-gas from the radiant section and by direct contact with steam. The vaporized feedstock and steam mixture is then introduced into the radiant section where the cracking takes place. The resulting stream having a temperature typically in the range of from 500 to 650° C. enters a fired tubular reactor and is heated to a temperature typically in the range of from 750 to 875° C. for 0.1 to 0.5 s, wherein the residence time, temperature profile and partial pressure is controlled. During this short reaction time, hydrocarbons in the feedstock are cracked into smaller molecules yielding light olefins such as ethylene, propylene, butylenes, other small olefins, and diolefins as major products besides methane. These reaction products typically leave the radiant tube at a temperature in the range of 800 to 850° C. and are preferably cooled to a temperature typically in the range of from 550 to 650° C. within 0.02 to 0.1 s in order to prevent degradation of the highly reactive compounds by secondary reactions. Then, the resulting stream leaves the furnace for further downstream processing.

In fluid catalytic cracking (FCC), a particulate catalyst, often having a particle size in the range of from 20 to 100 μm, circulates between a cracking reactor and a catalyst regenerator. In the reactor, a hydrocarbon feed contacts the hot, regenerated catalyst. The hot catalyst vaporizes and cracks the feed typically at 425 to 600° C. The cracking reaction deposits carbonaceous hydrocarbons, which eventually turn to coke on the catalyst, thereby deactivating it. The cracked products are separated from the coked catalyst, usually with the aid of a catalyst stripper, and the stripped catalyst is then regenerated within the regenerator. A catalyst regenerator burns coke from the catalyst with oxygen containing gas, usually air. Regeneration of the catalyst by oxidation restores catalyst activity and simultaneously typically heats the catalyst to 500 to 900° C. The heated catalyst is recycled to the cracking reactor to crack more fresh hydrocarbon feed.

According to the present invention, the cracking unit U_{CC} is preferably a thermal cracking unit, more preferably a steam cracking unit.

### Feed Streams S_{F}

The one or more hydrocarbon feed streams S_{F} of which at least one is passed into U_{C} and subjecting therein to hydrocarbon conversion according to (ii) may, for example, originate from upstream refinery processes such as an atmospheric distillation tower, a hydrocracker, a coker or the like and typically contains naphtha, liquefied petroleum gas (LPG), ethane, propane and/or butane. Alternatively or additionally, the one or more hydrocarbon feed streams S_{F} comprise at least one of a hydrocarbon feed stream comprising, preferably consisting of, fossil hydrocarbons; a hydrocarbon feed stream comprising, preferably consisting of, recycled hydrocarbons; and a hydrocarbon feed stream comprising, preferably consisting of, biohydrocarbons.

The term “fossil hydrocarbons” as used herein encompasses, for example, natural gas such as a naturally occurring mixture of gaseous hydrocarbons which primarily consists of methane in addition to small amounts of other higher alkanes such as ethane, propane and the like. Further, the term “fossil hydrocarbons” as used herein encompasses naphtha such as liquid hydrocarbon mixtures produced from natural gas condensates, petroleum distillates, and the distillation of coal tar and peat. Yet further, the term “fossil hydrocarbons” as used herein encompasses liquefied petroleum gas (LPG) such as a fuel gas containing a flammable mixture of hydrocarbon gases, in particular propane and butane, prepared by refining petroleum or “wet” natural gas.

The term “recycled hydrocarbons” as used herein encompasses, for example, pyrolysis oils obtained by pyrolysis of recycled plastic waste materials.

The term “biohydrocarbons” as used herein encompasses, for example, bio-based gases such as mixtures of gases primarily consisting of methane besides carbon dioxide and hydrogen sulfide which are produced from raw materials such as agricultural waste, manure, municipal waste, plant material, sewage, green waste, food waste and the like. Further, the term “biohydrocarbons” as used herein encompasses bio-naphtha such as naphtha produced from complex mixtures of naturally occurring fats and oils. Yet further, the term “biohydrocarbons” as used herein encompasses bio-liquefied petroleum gas (bio-LPG) such as liquefied petroleum gas produced from complex mixtures of naturally occurring fats and oils.

According to the present invention, the one or more hydrocarbon feed streams S_{F} preferably comprise at least one of a hydrocarbon feed stream comprising, preferably consisting of, fossil hydrocarbons; a hydrocarbon feed stream comprising, preferably consisting of, recycled hydrocarbons; and a hydrocarbon feed stream comprising, preferably consisting of, biohydrocarbons. Thus, also mixtures of two or more of the aforementioned are included.

Further according to the present invention, the unit U_{C} comprises heating means M_{H} for providing heat to the hydrocarbon conversion. Said heating means M_{H} comprise means for generating heat from one or more heat sources H_{S}, either from one or more plant-external sustainable heat sources H_{EXT-S}, and/or one or more plant-external fossil heat sources H_{EXT-F}, and/or one or more plant-internal heat sources H_{INT}. Further, the at least one heat source H_{S} comprises hydrogen (H₂) which is either comprised in H_{EXT-S}, and/or in H_{EXT-F} and/or in H_{INT}. Thus, the heating means M_{H} comprised in U_{C} comprise at least means for combusting H₂ and providing the resulting heat to the hydrocarbon conversion.

### Heat Demand of U_{C}

According to (i), one or more heat sources according to (3) are passed from M_{E} to M_{H} in an amount sufficient for generating heat in M_{H} meeting the heat demand E_{UC} of the hydrocarbon conversion in U_{C}.

According to the present invention, the heat demand E_{UC} of the hydrocarbon conversion process carried out in the unit U_{C} of the integrated plant is generally supplied via the one or more plant-external sustainable heat sources H_{EXT-S}. In case, however, the heat demand E_{UC} cannot be supplied by H_{EXT-S} alone, additional heat is required which, according to the present invention, is provided at least partially via the heat source H₂ comprised in the stream S_{H} obtained from the processing unit U_{P}. In addition to H₂ comprised in the stream S_{H}, also one or more other plant-internal heat sources H_{INT} may be used for supplying heat to U_{C} via the controllable means M_{E}, for example other sources of plant-internally product hydrogen and/or other plant-internally produced fuels and/or electrical heat which may be used. Preferably according to the present invention, the heat source H₂ comprised in the stream S_{H} is the sole plant-internal heat source H_{INT} which is used instead of or in addition to H_{EXT-S} for meeting the heat demand E_{UC} of U_{C}. If, and only if, the heat demand E_{UC} of U_{C} cannot be met by H_{EXT-S} in combination with H_{INT}, the remaining energy demand would be provided by one or more plant-external fossil heat sources H_{EXT-F}.

For combustion of hydrogen in M_{H}, the stream S′_{H}, based on the stream S_{H} and provided via the means M_{E} is preferably mixed with oxygen and combusted in burners or heating coils comprised in U_{C}.

Generally, there are no specific restrictions which sustainable heat source H_{EXT-S} is employed. Preferably, according to the present invention, the one or more plant-external sustainable heat sources H_{EXT-S} according to (3) comprise one or more of solar energy, wind energy, hydro energy, tidal energy, geothermal energy, biomass energy, ammonia obtained from renewable sources, biomethane, bio-LNG and H₂ obtained plant-externally from renewable sources. Depending on their respective physical nature, the plant-external sustainable heat sources H_{EXT-S} are, for example, used to provide heat via direct electrical heating or via combustion, as for example in the case of hydrogen wherein thermal energy is provided to the heat-consuming hydrocarbon conversion process. Generally, combusting hydrogen yields water as the only by-product. Thus, advantageously, in particular if hydrogen is employed as plant-external sustainable heat source H_{EXT-S}, the process of the present invention allows for an emission-reduced operation of the hydrocarbon conversion process, preferably the cracking process. As used herein, the term “emission-reduced operation” relates to a process which is carried out in such a way that the emission of greenhouse gases such as carbon dioxide is avoided or at least reduced, i.e. at a reduced carbon footprint.

### Processing of the Cracked Gas

In particular in the case when the unit U_{C} comprises a cracking unit U_{CC}, the gas stream obtained directly from the hydrocarbon conversion, i.e. the cracking process, said stream being referred to herein as the cracked gas stream S_{C}, is subjected to a one or more downstream process stages from which, among others the one or more product stream S and the light hydrocarbon conversion off-gas stream S_{O} are obtained.

Generally, the stream Sc which comprises light olefins such as ethylene, propylene, butylenes, other lower olefins and diolefins besides methane is separated into several different streams by using a sequence of separation and chemical-treatment stages. In the hydrocarbon conversion process, also light side products such as hydrogen, carbon oxides, light saturated hydrocarbons, and water are typically obtained. Suitably, the one or more product streams S, especially ethylene and propylene, are either used directly in downstream processes, optionally or preferably part of the integrated plant, or stored in storage vessels for subsequent use or long-term storage, either plant-internally or plant-externally.

The recovery of the various olefin products, i.e. the product streams, from cracked gas is usually carried out by fractional distillation using a series of distillation steps to separate out the various components. The unit which separates hydrocarbons with one carbon atom (C₁) and lighter fraction is generally referred to as “demethanizer”. The unit which separates hydrocarbons with two carbon atoms (C₂) from the heavier components is referred to as “deethanizer”. The unit which separates the hydrocarbon fraction with three carbon atoms (C₃) from the heavier components is referred to as “depropanizer”. The unit which separates the hydrocarbon fraction with four carbon atoms (C₄) from the heavier components is referred to as “debutanizer.”

The residual heavier components having a higher carbon number fraction (C₅₊) may be used as gasoline or be recycled back to the cracker. Alternatively, they may be sent to a suitable hydrocarbon-to-hydrogen conversion process.

The various fractionation units may be arranged in a variety of sequences in order to provide desired results based upon various feedstocks. To that end, a sequence which uses the demethanizer first is commonly referred to as the “front-end demethanizer” sequence. Similarly, when the deethanizer is used first, it is commonly referred to as the “front-end deethanizer” sequence. And, when the depropanizer is used first, it is commonly referred to as “front-end depropanizer” sequence.

In the conventional front-end demethanizer sequence, the cracked gas containing hydrocarbons having one to five or more carbon atoms per molecule (C₁ to C₅₊) first enters a demethanizer, where methane and lighter fractions (hydrogen) are separated as an over-head stream. The demethanizer usually operates at relatively low temperatures, typically in the range of from −100° C. to about 25° C.

The front-end demethanizer over-head stream constitutes a generally suitable light hydrocarbon off-gas stream to be passed to the methane pyrolysis unit U_{M} according to (iii). Alternatively, hydrogen contained in the front-end demethanizer over-head stream may be removed first and the remaining gas consisting mainly of methane is passed as the stream S_{O} to the methane pyrolysis unit U_{M} according to (iii).

The heavy ends leaving the demethanizer consist mainly of C₂ to C₅₊ molecules. These heavy ends then are preferably passed to a deethanizer where the C₂ hydrocarbons are removed at the top and the C₃ to C₅₊ compounds leave the deethanizer as bottoms. The C₂ components leaving the top of the deethanizer may be fed to an acetylene converter or acetylene removal unit. As some methane remains dissolved in the heavy ends exiting the demethanizer and ends up in the C₂ components leaving the deethanizer, the C₂ components stream may be subsequently sent to a demethanizer for removal of the remaining methane. This residual demethanizer over-head stream then would constitutes a suitable light hydrocarbon off-gas stream S_{O} to be passed to the methane pyrolysis unit U_{M} according to (iii).

Therefore, the unit U_{C} preferably further comprises a separation unit U_{S}, wherein according to
(ii), the process comprises
(ii.1) passing at least one hydrocarbon feed stream S_{F} into U_{C} and subjecting the at least one feed stream S_{F} to hydrocarbon conversion in U_{C}, obtaining a cracked gas stream S_{C};
(ii.2) passing the cracked gas stream S_{C} obtained according to (ii.1) to a separation unit U_{S} comprising at least one demethanizing unit, obtaining from said demethanizing unit the stream S_{O}, wherein from U_{S}, the one or more product streams S are obtained.

As described above, the separation unit U_{S} preferably comprises a distillation tower from which an overhead stream is obtained which comprises methane and hydrogen. The overhead stream comprises methane and hydrogen as the main components. The ratio of methane and hydrogen in the overhead stream may vary depending on the cracking operation conditions, and the hydrocarbon feed streams S_{F}, respectively. Reference can be made, for example, to Ullmann's Encyclopedia of Industrial Chemistry, Ethylene 5.1.3 Commercial Cracking Yields, DOI: 10.1002/14356007.a10_045.pub3 for different cracking yields depending on different cracker feedstocks. Generally, the methane concentration in the overhead is in the range of from 40 to 95 weight-%, preferably in the range of from 90 to 95 weight-% of methane, with the remainder being mainly hydrogen.

Still further preferred is a separation unit which, in addition to and downstream of said distillation tower, comprises a gas separation unit from which, based on said overhead stream comprising methane and hydrogen, a methane-rich stream and a hydrogen-rich stream are obtained.

Therefore, the demethanizing step according to (ii.2) preferably comprises a distillation step from which an overhead stream S_{O1} is obtained comprising CH₄ and H₂, and further preferably, the demethanizing step according to (ii.2) comprises a separation step wherein S_{O1} is separated into a H₂-rich stream and a CH₄-rich stream S_{O2}, wherein S_{O1} or a partial stream thereof, preferably S_{O2} or a partial stream thereof, is subjected as the stream S_{O} into U_{M} according to (iii).

As far as the separation step wherein S_{O1} is separated into a H₂-rich stream and a CH₄-rich stream S_{O2} is concerned, no particular limitations exist regarding the suitable unit in which this separation is carried out. As an example, said separation can be carried out using a pressure swing adsorption unit. Preferably, the H₂-rich stream exhibits a H₂ content in the range of from 90 to 100 volume-%, more preferably in the range of from 95 to 100 volume-%. Optionally, according to the present invention, this H₂-rich stream can be suitably combined with the stream S′_{H} in which case according to (c.1), the determination of the amount of H₂ necessary to produce heat in M_{H} in an amount of ΔE and controlling the supply means M_{E} and optionally the dividing means M_{D} so that an amount of H₂ is passed into My via S_{H} which is sufficient to produce ΔE in M_{H} would take into the H₂ content of said this H₂-rich stream.

As far as the composition of the stream S_{O} to be passed to U_{M} is concerned, it is preferred that from 96 to 100 volume-% of S_{O} consist of CH₄. More preferably from 97 to 100 volume-%, more preferably from 98 to 100 volume-%, more preferably from 99 to 100 volume-%, more preferably from 99.5 to 100 volume-%, more preferably from 99.9 to 100 volume-% of S_{O} consists of CH₄. Further preferably, from 0 to 4 volume-% of S_{O} consist of H₂. More preferably from 0 to 3 volume-%, more preferably from 0 to 2 volume-%, more preferably from 0 to 1 volume-%, more preferably from 0 to 0.5 volume-%, more preferably from 0 to 0.1 volume-% of S_{O} consist of H₂.

The C₂ components from which methane has been removed are then preferably sent to a C₂ splitter which produces ethylene as the light product and ethane as the heavy product. The C₃ to C₅₊ stream leaving the bottom of the deethanizer is preferably routed to a depropanizer from which the C₃ components are obtained overhead and the C₄ to C₅₊ components are obtained as the bottoms. The C₃ product may be hydrotreated to remove C₃ acetylene and dienes before being fed to a C₃ splitter where it is separated into propylene at the top and propane at the bottom. The C₄ to C₅₊ stream is preferably fed to a debutanizer from which C₄ components are obtained at the top with the balance of C₅₊ components being obtained as the bottoms. Both the C₄ and the C₅₊ streams may be separately hydrotreated to remove undesirable acetylenes and dienes.

In conventional front-end deethanizer sequences, the cracked gas Sc containing C₁ to C₅₊ components first enters a deethanizer. The light ends exiting the deethanizer consist of C₂ and C₁ components along with any hydrogen. These light ends are usually fed to a demethanizer where the hydrogen and C₁ are removed as light components and the C₂ components are removed as the heavy components. The C₂ stream leaving the bottom of the demethanizer may be fed to an acetylene converter and then to a C₂ splitter which produces ethylene as the light product and ethane as the heavy product. The heavies leaving the deethanizer which consist of C₃ to C₅₊ components are usually routed to a depropanizer from which the C₃ components are obtained overhead and the C₄ to C₅₊ are obtained as the bottoms. The C₃ product is the usually fed to a C₃ splitter where it is separated into propylene at the top and propane at the bottom, while the C₄ to C₅₊ stream is fed to a debutanizer which produces C₄ compounds at the top with the balance leaving as bottoms to be used for gasoline or to be recirculated as feed into the cracking process. As with the front-end demethanizer sequence, the C₃, C₄, and C₅₊ streams may be separately hydrotreated to remove undesirable acetylenes and dienes.

In conventional front-end depropanizer sequences, the quenched and acid-free gases containing hydrocarbons having from one to five or more carbon atoms per molecule (C₁ to C₅₊) first enter a depropanizer. The heavies leaving the depropanizer consist of C₄ to C₅₊ components. These are usually passed to a debutanizer where the C₄ components and lighter species are taken over the top with the rest of the feed leaving as bottoms which can be used for gasoline or other chemical recovery. These streams may be separately hydrotreated to remove undesired acetylenes and dienes. The lights of the depropanizer containing C₁ to C₃ components may be fed to an acetylene converter and then to a demethanizer system where the C₁ components and any remaining hydrogen are generally removed overhead. The heavies leaving the demethanizer system containing C₂ and C₃ components are usually passed into a deethanizer wherein C₂ components are removed from the top and C₃ compounds are obtained as the bottoms. The C₂ components are, in turn, usually fed to a C₂ splitter which produces ethylene as the light product and ethane as the heavy product. The C₃ stream is fed to a C₃ splitter which separates the C₃ species, sending propylene to the top and propane to the bottom.

As with the front-end demethanizer sequence, the saturated C₂ hydrocarbons and/or the saturated C₃ hydrocarbons obtained in the front-end deethanizer sequence or the front-end depropanizer sequence or a partial stream thereof may be recycled as feed into the cracking process.

### The Methane Pyrolysis Unit U_{M}

According to the present invention, the light hydrocarbon off-gas stream S_{O} is passed according to (iii) into the methane pyrolysis unit U_{M} according to (4) where it is subjected to methane pyrolysis and wherein from U_{M}, the H₂-rich gas stream S_{H} is obtained.

Methane pyrolysis is a technology according to which hydrocarbons, especially aliphatic hydrocarbons such as methane, ethane, propane and/or butane are split directly into hydrogen and solid carbon. For methane, the pyrolysis proceeds according to the following main reaction:

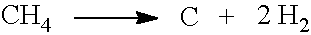

During methane pyrolysis, the release of greenhouse gases is prevented. Therefore, in the event that the energy used for carrying out the pyrolysis reaction originates from renewable resources, methane pyrolysis is a CO₂-free, i.e. clean technology to obtain emission-free hydrogen. Methane pyrolysis is a one-step process which produces hydrogen in high volume.

Preferably, a reaction zone in which methane pyrolysis is carried out is operated at a temperature in the range of from 800 to 1500° C. and at a residence time of at least 1 s. Carrying out the methane pyrolysis at said reaction conditions advantageously allows for obtaining solid carbon besides a hydrogen-containing stream which is essentially free of impurities. In this context, the term “essentially free” denotes that the hydrogen-containing stream comprises only trace amounts of impurities such as methane and/or polycyclic carbonaceous species. Preferably, from 85 to 100 volume-%, more preferably from 90 to 98 volume-%, more preferably from 90 to 96 volume-% of the hydrogen-containing stream consist of hydrogen.

A further advantage of methane pyrolysis is that the obtained solid carbon can be sold as a commercial product for selected applications, depending on the carbon morphology and physical/chemical properties. For example, the solid carbon from methane pyrolysis may be used for aluminum and steel production, tire manufacturing, electrode manufacturing, polymer blending, additive for construction materials, carbon devices like heat exchangers, soil conditioning, or storage.

Methane pyrolysis can be conducted in different ways generally known to the persons skilled in the art: catalytically or thermally, with heat input via plasma, resistance heating, liquid metal or micro-wave processes or autothermally. In this respect, reference is made, for example, to N. Muradov and T. Veziroglu: “Green” path from fossil-based to hydrogen economy: An overview of carbon-neutral technologies”, International Journal Hydrogen Energy 33 (2008) 6804-6839; H. F. Abbas and W. M. A. Wan Daud: Hydrogen production by methane decomposition: A review, International Journal Hydrogen Energy 35 (2010) 1160-1190); R. Dagle et al.: An Overview of Natural Gas Conversion Technologies for Co-Production of Hydrogen and Value-Added Solid Carbon Products, Report by Argonne National Laboratory and Pacific Northwest National Laboratory (ANL-17/11, PNNL-26726) November 2017.

For example, according to the present invention, the methane pyrolysis can be carried out by subjecting the stream S_{O} to decomposition and deposition of the carbon comprised in the stream S_{K} on suitable underlying substrates (carbon materials, metals, ceramics and mixtures thereof), preferably at temperatures in the range of from 1000 to 2500 K and at pressures in the range of from 0.5 to 10000 kPa (abs). The substrate can either be porous or non-porous and can either be a support substrate in the reactor (a pre-installed part) or a granular and powder material. The particle size of a preferred support substrate is generally in the range of from 0.3 to 15 mm, preferably in the range of from 0.5 to 10 mm, more preferably in the range of from 1 to 8 mm, more preferably in the range of from 3 to 8 mm. The decomposition can either be realized using a fixed bed, a moving carbon bed, a fluidized bed or entrained flow.

As the energy demand of the methane pyrolysis contributes to the energy-penalty of the carbon fixture, a methane pyrolysis process having a high as possible energy efficiency is preferred. Among the above-mentioned examples, moving carbon bed methane pyrolysis is preferred due to its low energy demand and its higher hydrogen yields as compared to the above-mentioned other methane pyrolysis methods. The methane pyrolysis is not limited to a specific energy supply. Preferably, the heat demand of the pyrolysis is provided via electrical heating.

A further alternative methane pyrolysis method for producing carbon black comprises generating a torch plasma by subjecting a plasma gas to a plasma arc, mixing the stream S_{O} with the plasma gas and combining the mixture in a reactor at a given reactor temperature to produce the stream S_{K} comprising carbon black, wherein the stream S_{O} is mixed with the plasma gas outside of the area occupied by the plasma arc.

A further alternative methane pyrolysis method for producing carbon black comprises introducing the stream S_{O} into a reaction space where the methane is thermally decomposed to carbon and hydrogen in the presence of carbonaceous pellets. In this process, a part of the required thermal energy is provided by means of a gaseous heat carrier which is produced outside the reaction space and then introduced into the reaction space. Upon introduction into the reaction zone, the gaseous heat carrier releases a part of its heat directly to the carbonaceous pellets and/or the methane to be decomposed.

A further alternative methane pyrolysis method is carried out in a liquid metal bubble column. This process advantageously gives rise to especially high methane conversions.

A further alternative of methane pyrolysis is carrying out the process in a molten medium. Doing so allows for superior heat management and temperature control. In addition, methane decomposition in a molten medium prevents deposition of solid carbon layers on the reactor wall which is the primary cause of reactor plugging.

Control of the carbon morphology may be achieved via chemical vapor deposition (CVD) methods. With a CVD method, a catalyst, which may be a metal, is exposed to gaseous hydrocarbons to produce high quality carbon materials on the catalyst surface. Supported metal catalysts can be employed in fluidized bed reactors.

In case the hydrogen-rich stream obtained from methane pyrolysis according to the present invention should contain any significant amounts of methane, it may be desirable to subject said hydrogen-rich stream, prior to passing it to M_{E}, to a suitable gas separation stage to separate methane from hydrogen. In these cases, according to which the methane pyrolysis unit U_{M} according to (4) comprises a gas separation unit U_{GS}, the process according to (iii) comprises
(iii.1) passing S_{O} into U_{M} and subjecting S_{O} to methane pyrolysis in U_{M}, obtaining a stream S_{HM} comprising methane and hydrogen, and obtaining the stream S_{K};
(iii.2) passing the stream S_{HM} into U_{GS}, obtaining a stream S_{M} being enriched in methane compared to S_{HM}, and obtaining the stream S_{H}.

The respectively obtained methane-rich stream S_{M} may be put to further use wherein it may be advantageous to recycle the stream S_{M} or a part stream thereof as feed stream to U_{M}, in addition to the respective feed stream S_{O}. By doing so, the net efficiency of the hydrogen production in U_{M} is increased. Preferably, the stream S_{M} which is obtained from the gas separation unit U_{GS} described above is passed through a controllable dividing means M_{D}, such as a controllable valve, from which a stream S′_{M} and a stream S″_{M} are obtained, preferably having the same chemical composition as S_{M}. Therefore, the present invention also relates to a method as described above, wherein the integrated plant further comprises controllable dividing means M_{D} arranged downstream of U_{M}, wherein the process according to (iii) further comprises
(iii.3) passing the stream S_{M} into the dividing means M_{D}, obtaining a stream S′_{M} being x volume-% of S_{M}, with 0≤x≤100, and obtaining a stream S″_{M} being (100-x) volume-% of S_{M}.

It is noted that according to (iii.3), it is possible to pass the stream S_{M} through M_{D} without obtaining a stream S″_{M}, i.e. no dividing take place and the stream S′_{M} is identical to the stream S_{M}. In this case, x=0. On the other hand, it is also possible to pass the stream S_{M} through M_{D} without obtaining a stream S′_{M}, i.e. no dividing take place and the stream S″_{M} is identical to the stream S_{M}. In this case, x=100. As noted above, if x≠0, it is possible to recycle the stream S′_{M}, and the process stage (iii) further comprises
(iii.4) passing the stream S′_{M} as feed stream into U_{M}.

### The Method of Controlling the Process

According to the method of the present invention, the process carried out in the integrated plant is controlled in a suitable manner so that the heat demand E_{UC} of the hydrocarbon conversion process carried out in the unit U_{C} can be met, in case the amount of heat which can be supplied by external and sustainable heat sources H_{EXT-S} is too low, at least partially by using hydrogen as a heat source for U_{C}, which hydrogen is comprised in a gas stream which in turn is suitably obtained from an off-gas stream of said unit U_{C}.

Further according to the present invention, this method comprises the determination of the heat demand E_{UC} of the hydrocarbon conversion carried out in the unit U_{C}. Depending on, e.g., the specific chemical composition of the at least one hydrocarbon feed stream S_{F} which is passed into U_{C} and/or the specific reaction conditions of the hydrocarbon conversion process carried out in U_{C} which composition and/or conditions may vary over time during the hydrocarbon conversion process, said heat demand E_{UC} may also vary over time. Yet further, as indicated above, the amount of heat which can be provided by the plant-external sustainable heat sources H_{EXT-S} may vary over time, e.g. due to general supply issues such as costs involved for purchasing suitable heat sources H_{EXT-S}, or supply interruptions due to maintenance intervals. All of these different possible influences can be dealt with by the control method of the present invention which renders said process highly flexible.

According to the method of the present invention according to (a), the heat demand of E_{UC} of U_{C} is suitably determined. This determination can be carried out continuously or semi-continuously at respective intervals which depend on the varying parameters having an impact on the hydrocarbon conversion process in U_{C} as described above. Further according to the method of the present invention according to (b), the amount E_{EXT-S} of heat available for U_{C} from the one or more plant-external sustainable heat sources H_{EXT-S} is determined continuously or semi-continuously at respective intervals which depend on the varying parameters having an impact on the hydrocarbon conversion process in U_{C} as described above. As a result from the values for E_{UC} and E_{EXT-S} determined according to (a) and (b), it is known at every desired point in time during the hydrocarbon process if the heat demand E_{UC} can be met by E_{EXT-S} by determining the value of ΔE=E_{UC}−E_{EXT}, i.e. by straight-forward calculation.

### ΔE>0

If the determination of ΔE as described above leads to a positive value, ΔE>0, at least a certain amount of additional plant-internal heat is necessary to suitably meet the energy demand of and, thus, to maintain the hydrocarbon conversion process in U_{C}.

In particular, according to the present invention, the amount of hydrogen H_{2N} is then determined which needs to be provided as heat source to M_{H} in order to produce heat in an amount of ΔE. Yet further, according to the present invention, the amount of hydrogen H_{2P} is then determined, the maximum amount of H₂ which can be produced in U_{M} and, thus, the maximum amount of H₂ which can be achieved in the stream S_{H}. Based this amount H_{2P}, heat can be produced in M_{H} in an amount of E_{H2P}.

### ΔE>0 and ΔH>0

If a comparison of the values H_{2N} and H_{2P} leads to the conclusion that ΔH=H_{2N}−H_{2P} is greater than zero, i.e. that the heat demand ΔE cannot be met by H_{2P} alone, it is particularly preferred that said maximum amount H_{2P} is passed via the stream S′_{H} into M_{H}. The supply means M_{E} are suitably controlled to provide the maximum amount H_{2P} to M_{H} via S′_{H} so that E_{H2P} can be produced in M_{H}. It is noted that it may be possible, also in this case where ΔH>0, not to pass the maximum amount H_{2P} into M_{H}, for example in case if other plant-internal sustainable heat sources might be available which could suitably replace a part of H_{2P} when passed into M_{H}; however, passing the maximum amount H_{2P} to M_{H} is generally particularly preferred.

According to this scenario, the process comprises
(iv) passing the stream S_{H} obtained from U_{M} through M_{E}, obtaining a stream S′_{H} being y volume-% of S_{H}, with y=100;
(v) passing the stream S′_{H} obtained from M_{E} as H_{INT} into M_{H}.

Since, as discussed above, the energy demand ΔE cannot be met by H_{2P} according to this process scenario, at least one additional heat source must be passed to M_{H} in order to produce sufficient heat to maintain the hydrocarbon conversion process. While it may be generally conceivable that said at least one additional heat source may be available plant-internally, the at least one additional heat source is usually provided via a plant-external fossil heat source. Therefore, the process preferably comprises, further to (iv) and (v) above,
(vi) passing at least one of the one or more plant-external fossil heat sources H_{EXT-F} via M_{E} into M_{H} in an amount sufficient to generate heat in an amount (ΔE−E_{H2P}) in M_{H}.

As mentioned above, in case the hydrogen-rich stream obtained from methane pyrolysis contains a certain amount of methane, the respective stream S_{HM} can be subjected to a gas separation in the unit U_{GS}, obtaining the stream S_{H} and the methane-rich stream S_{M}. In particular in case when ΔE>0 and ΔH>0, this stream S_{M} or a part thereof can be recycled back via the stream S′_{M} as feed stream to U_{M} since by doing so, the net efficiency of the methane pyrolysis will be increased. In this case, the controllable means M_{D} can be controlled in such a manner that the amount of the recycle stream S′_{M} is respectively set to provide a gas stream S_{H} containing the maximal producible of H₂, H_{2P}.

### ΔE>0 and ΔE≤0

If a comparison of the values H_{2N} and H_{2P} leads to the conclusion that ΔH=H_{2N}−H_{2P} is less than or equal to than zero, i.e. that the heat demand ΔE can be met by H_{2P} alone, the supply means M_{E} are suitably controlled to provide the necessary amount of plant-internal H₂ to M_{H} via S′_{H} so that ΔE can be produced in M_{H}.

According to this scenario, the process comprises
(iv′) passing the stream S_{H} obtained from U_{M} through M_{E}, obtaining a stream S′_{H} being y volume-% of S_{H}, with 0<y≤100;
(v′) passing the stream S′_{H} obtained from M_{E} as H_{INT} into M_{H};

If, for example, the entire stream S_{H} is needed to supply the necessary heat demand to U_{C}, the means M_{E} are controlled so that the entire stream S_{H} is passed as stream S′_{H} to M_{H}. In this case, y=100.

If only a part of the stream S_{H} is needed to supply the necessary heat demand to U_{C}, the means M_{E} are controlled so that a respective part of S_{H} containing the determined amount of hydrogen is passed as stream S′_{H} to M_{H}. In this case, the process comprises
(iv′) passing the stream S_{H} obtained from U_{M} through M_{E}, obtaining a stream S′_{H} being y volume-% of S_{H}, with 0<y<100, and obtaining a stream S″_{H} being (100-y) volume-% of S_{H};
(v′) passing the stream S′_{H} obtained from M_{E} as H_{INT} into M_{H}.

The stream S″_{H} or a part thereof is then preferably put to further use, wherein said further use preferably comprises the use of S″_{H} or the part thereof as a heat source in one or more units other than U_{C}. More preferably, in said one or more units other than U_{C}, S″_{H} or a part stream thereof is used as a heat source for one or more endothermic processes carried out at a temperature of at least 800° C., for the generation of steam or generally for combustion.

If the stream S_{H} which is passed into M_{E} and separated there in a stream S′_{H} to be passed to M_{H} and a stream S″_{H}, said separation can be carried out, for examples, using one or more controllable valves.

As mentioned above, in case the hydrogen-rich stream obtained from methane pyrolysis contains a certain amount of methane, the respective stream S_{HM} can be subjected to a gas separation in the unit U_{GS}, obtaining the stream S_{H} and the methane-rich stream S_{M}. In particular in case when ΔE>0 and ΔH≤0, this stream S_{M} or a part thereof can be recycled back via the stream S′_{M} as feed stream to U_{M} since by doing so, the net efficiency of the methane pyrolysis will be increased. In this case, based on the specific value of ΔE and the respectively determined amount of H₂ which needs to be comprised in the stream S_{H}, the controllable means M_{D} can be controlled in such a manner that the amount of the recycle stream S′_{M}, i.e. the value of x, is respectively set to provide a gas stream S_{H} containing at least the desired amount of H₂.

Preferably according to the present invention, from 60 to 100 volume-%, more preferably from 70 to 100 volume-%, more preferably from 80 to 100 volume-%, more preferably from 90 to 100 volume-%, more preferably from 95 to 100 volume-%, more preferably from 99 to 100 volume-% of the H₂-rich stream S_{H} consist of H₂.

### ΔE≤0

If the determination of ΔE leads to a non-positive value, ΔE≤0, then it is known that no additional plant-internal heat is necessary to maintain the hydrocarbon conversion process in U_{C}. In this scenario, the supply means M_{E} are controlled in such a manner so that no H₂ is passed into M_{H} via S_{H}. Thus, no stream S′_{H} is supplied by M_{E} to M_{H}, and the means M_{E} are controlled in such a manner the entire stream S_{H} is obtained as the stream S″_{H}.

In this case, it is preferred that the stream S″_{H} or a part thereof is put to further use, wherein said further use preferably comprises the use of S″_{H} as a heat source in one or more units other than U_{C}. More preferably, in said one or more units other than U_{C}, S″_{H} or a part stream thereof is used as a heat source for one or more endothermic processes carried out at a temperature of at least 800° C., for the generation of steam or generally for combustion.

In particular in case the maximum amount H_{2P} shall be passed via M_{E} to M_{H}, it may be desirable to pass the stream S′m as feed stream into U_{M} according to (iii.4) as described above with the proviso that the methane pyrolysis unit U_{M} according to (4) comprises a gas separation unit U_{GS} and the process according to (iii) comprises
(iii. 1) passing S_{O} into U_{M} and subjecting S_{O} to methane pyrolysis in U_{M}, obtaining a stream S_{HM} comprising methane and hydrogen, and obtaining the stream S_{K};
(iii.2) passing the stream S_{HM} into U_{GS}, obtaining a stream S_{M} being enriched in methane compared to S_{HM}, and obtaining the stream S_{H};
(iii.3) passing the stream S_{M} into the dividing means M_{D}, obtaining a stream S′_{M} being x volume-% of S_{M}, with 0<x≤100, and obtaining a stream S″_{M} being (100-x) volume-% of S_{M};

Preferably, the method of controlling the process according to the present invention makes use of a computer-supported control system with which one or more of the above-described parameters are determined and one or more of the above-described units and/or means and/or processes are controlled based on said one or more parameters in order to achieve the desired process design.

Still further, the present invention relates to an integrated production plant, comprising
(1) supply means M_{F} for providing one or more hydrocarbon feed streams S_{F} to a unit U_{C} for heat-consuming hydrocarbon conversion, wherein the supply means M_{F} are connected to U_{C} for passing at least one of said hydrocarbon feed stream S_{F} into U_{C} to obtain from U_{C} one or more product streams S and a light hydrocarbon conversion off-gas stream S_{O} comprising CH₄;
(2) the unit U_{C} which comprises heating means M_{H} for providing heat to the hydrocarbon conversion, wherein said heating means M_{H} comprise means for generating heat from one or more heat sources H_{S} and wherein said one or more heat sources H_{S} comprise H₂;
(3) controllable supply means M_{E} for providing one or more plant-external sustainable heat sources H_{EXT-S} one or more plant-external sustainable fossil heat sources H_{EXT-F} and one or more plant-internal heat sources H_{INT}, said plant-internal heat sources H_{INT} comprising H₂, wherein the supply means M_{E} are connected to U_{C} for passing at least one of said heat sources H_{EXT-S}, H_{EXT-F} and H_{INT} to the heating means M_{H};
(4) a methane pyrolysis unit U_{M} for subjecting the light hydrocarbon conversion off-gas stream S_{O} to methane pyrolysis to obtain a hydrogen-rich gas stream S_{H} and a carbon stream S_{K};
(5) optionally a gas separation unit U_{GS} comprised in U_{M} for separating a gas stream S_{M} obtained from methane pyrolysis into the stream S_{H} and a stream S_{M} being enriched in methane compared to S_{HM};
(6) and optionally controllable dividing means M_{D} for optionally dividing the stream S_{M} into a part stream S′_{M} and a stream S″_{M}, wherein M_{D} is connected to U_{M} for passing S_{M} into M_{D} and passing S′_{M} into U_{M}.

Preferably, the unit U_{C} for heat-consuming hydrocarbon conversion comprises a cracking unit U_{CC}, preferably a thermal cracking unit, more preferably a steam cracking unit. More preferably, the unit U_{C} further comprises a separation unit U_{S} for treating a cracked gas stream S_{C}, wherein U_{S} comprises at least one demethanizing unit, wherein from said demethanizing unit the stream S_{O} is obtained and wherein from U_{S}, the one or more product streams are obtained.

Still further, the present invention also relates to the use of the integrated production plant as described above for carrying out the method as described above.

The present invention is further illustrated by the following set of embodiments and combinations of embodiments resulting from the dependencies and back-references as indicated. In particular, it is noted that in each instance where a range of embodiments is mentioned, for example in the context of a term such as “The method of any one of embodiments 1 to 4”, every embodiment in this range is meant to be explicitly disclosed for the skilled person, i.e. the wording of this term is to be understood by the skilled person as being synonymous to “The method of any one of embodiments 1, 2, 3 and 4”. Further, it is explicitly noted that the following set of embodiments represents a suitably structured part of the general description directed to preferred aspects of the present invention, and, thus, suitably supports, but does not represent the claims of the present invention.
1. A method for controlling a process carried out in an integrated production plant, wherein the integrated production plant comprises
   (1) supply means M_{F} for providing one or more hydrocarbon feed streams S_{F} to a unit U_{C} for heat-consuming hydrocarbon conversion to obtain from U_{C} one or more product streams S and a light hydrocarbon conversion off-gas stream S_{O} comprising CH₄;
   (2) the unit U_{C} which comprises heating means M_{H} for providing heat to the hydrocarbon conversion, wherein said heating means M_{H} comprise means for generating heat from one or more heat sources H_{S} and wherein said one or more heat sources H_{S} comprise H₂;
   (3) controllable supply means M_{E} for providing one or more plant-external sustainable heat sources H_{EXT-S}, one or more plant-external fossil heat sources H_{EXT-F}, and one or more plant-internal heat sources H_{INT}, said plant-internal heat sources H_{INT} comprising H₂, for passing at least one of said heat sources H_{EXT-S}, H_{EXT-F} and H_{INT} to the heating means M_{H};
   (4) a methane pyrolysis unit U_{M} for subjecting the light hydrocarbon conversion off-gas stream S_{O} to methane pyrolysis to obtain a hydrogen-rich gas stream S_{H} and a carbon stream S_{K};
   wherein the process comprises
   (i) passing one or more heat sources according to (3) from M_{E} to M_{H} in an amount sufficient for generating heat in M_{H} meeting the heat demand E_{UC} of U_{C};
   (ii) passing at least one hydrocarbon feed stream S_{F} into U_{C} and subjecting the at least one feed stream S_{F} to hydrocarbon conversion in U_{C}, obtaining the one or more product streams S and S_{O};
   (iii) passing S_{O} into U_{M} and subjecting S_{O} to methane pyrolysis in U_{M}, obtaining S_{H} and S_{K};
   wherein the method for controlling the process comprises
   (a) determining the heat demand E_{UC} of the hydrocarbon conversion in U_{C} to be provided by M_{H};
   (b) determining the amount E_{EXT-S} of heat available for U_{C} from the one or more plant-external sustainable heat sources H_{EXT-S};
   (c) determining ΔE=E_{UC}−E_{EXT-S} and

| (c.1) | if ΔE > 0, | |
|---|---|---|
| | (c.1.1) | determining H_{2N}, the amount of H₂ necessary to produce ΔE in M_{H}; |
| | (c.1.2) | determining H_{2P}, the maximum amount of H₂, producible in U_{M} to be |
| | | contained in S_{H}, to produce an amount E_{H2P} of heat in M_{H}; |
| | (c.1.3) | determining ΔH = H_{2N} − H_{2P}; and |

| | |
|---|---|
| (c.1.3.1) | if ΔH > 0, |
| | controlling the supply means M_{E} so that H_{2P} is passed into |
| | M_{H} via S_{H}; |
| | wherein the process further comprises |

| | |
|---|---|
| (iv) | passing the stream S_{H} obtained from U_{M} through |
| | M_{E}, obtaining a stream S′_{H} being y volume-% of S_{H}, |
| | with y = 100; |
| (v) | passing the stream S′_{H} obtained from M_{E} as H_{INT} |
| | into M_{H}; |
| (vi) | passing at least one of the one or more plant- |
| | external fossil heat sources H_{EXT-F} via M_{E} into M_{H} in |
| | an amount sufficient to generate heat in an amount |
| | (ΔE − E_{H2P}) in M_{H}; |

| | |
|---|---|
| (c.1.3.2) | if ΔH ≤ 0, |
| | controlling the supply means M_{E} so that an amount of H₂ |
| | is passed into M_{H} via S_{H} which is sufficient to produce ΔE |
| | in M_{H}; |
| | wherein the process further comprises |

| | |
|---|---|
| (iv′) | passing the stream S_{H} obtained from U_{M} through |
| | M_{E}, obtaining a stream S′_{H} being y volume-% of S_{H}, |
| | with 0 < y ≤ 100; |
| (v′) | passing the stream S′_{H} obtained from M_{E} as H_{INT} |
| | into M_{H}; |

| (c.2) | if ΔE ≤ 0, | |
|---|---|---|
| | controlling the supply means M_{E} so that no H₂ is passed into M_{H} via S_{H}; | |
| | wherein the process further comprises | |
| | (iv″) | passing the stream S_{H} through M_{E}, obtaining a stream S″_{H} being 100 |
| | | volume-% of S_{H}; |
| | (v″) | removing the stream S″_{H} from M_{E}. |

2. The method of embodiment 1, wherein from 40 to 100 volume-%, preferably from 60 to 100 volume-%, more preferably from 80 to 100 volume-%, more preferably from 90 to 100 volume-%, more preferably from 95 to 100 volume-% of S_{O} consist of CH₄.
3. The method of embodiment 1 or 2, wherein the unit U_{C} for heat-consuming hydrocarbon conversion comprises a cracking unit U_{CC}, preferably a thermal cracking unit, more preferably a steam cracking unit.
4. The method of embodiment 3, wherein U_{C} further comprises a separation unit Us, wherein according to (ii), the process comprises
   (ii.1) passing at least one hydrocarbon feed stream S_{F} into U_{C} and subjecting the at least one feed stream S_{F} to hydrocarbon conversion in U_{C}, obtaining a cracked gas stream Sc;
   (ii.2) passing the cracked gas stream S_{C} obtained according to (ii.1) to a separation unit Us comprising at least one demethanizing unit, obtaining from said demethanizing unit the stream S_{O}, wherein from U_{S}, the one or more product streams S are obtained.
5. The method of embodiment 4, wherein the demethanizing step according to (ii.2) comprises a distillation step from which an overhead stream S_{O1} is obtained comprising CH₄ and H₂, and preferably further comprises a separation step wherein S_{O1} is separated into a H₂-rich stream and a CH₄-rich stream S_{O2}, wherein S_{O1} or a partial stream thereof, preferably S_{O2} or a partial stream thereof, is subjected as the stream S_{O} into U_{M} according to (iii).
6. The method of any one of embodiments 1 to 5, preferably of any one of embodiments 3 to 5, wherein from 96 to 100 volume-%, preferably from 98 to 100 volume-%, more preferably from 99 to 100 volume-%, more preferably from 99.9 to 100 volume-% of S_{O} consists of CH₄.
7. The method of any one of embodiments 1 to 6, preferably of embodiment 6, wherein from 0 to 4 volume-%, preferably from 0 to 2 volume-%, more preferably from 0 to 1 volume-%, more preferably from 0 to 0.1 volume-% of S_{O} consist of H₂.
8. The method of any one of embodiments 1 to 7, wherein the one or more hydrocarbon feed streams S_{F} comprise at least one of a hydrocarbon feed stream comprising, preferably consisting of, fossil hydrocarbons; a hydrocarbon feed stream comprising, preferably consisting of, recycled hydrocarbons; and a hydrocarbon feed stream comprising, preferably consisting of, biohydrocarbons.
9. The method of any one of embodiments 1 to 8, wherein the one or more plant-external sustainable heat sources H_{EXT-S} according to (3) comprise one or more of solar energy, wind energy, hydro energy, tidal energy, geothermal energy, and biomass energy, ammonia obtained from renewable sources, biomethane, bio-LNG and H₂ obtained from renewable sources, wherein the plant-external fossil heat sources H_{EXT-F} according to (3) preferably comprise one or more of coal, oil and gas.
10. The method of any one of embodiments 1 to 9, wherein the carbon comprised in S_{K} is stored or put to further use, preferably as a component for producing one or more of tyres, for producing of one or more of aluminium and steel, for preparing printing inks, for preparing electrodes, for polymer blending, as additive for construction materials, or for soil improvement.
11. The method of any one of embodiments 1 to 10, wherein the methane pyrolysis unit U_{M} according to (4) comprises a gas separation unit UCs, wherein the process according to (iii) comprises
   (iii.1) passing S_{O} into U_{M} and subjecting S_{O} to methane pyrolysis in U_{M}, obtaining a stream S_{HM} comprising methane and hydrogen, and obtaining the stream S_{K};
   (iii.2) passing the stream S_{HM} into U_{GS}, obtaining a stream S_{M} being enriched in methane compared to S_{HM}, and obtaining the stream S_{H}.
12. The method of embodiment 11, wherein the integrated plant further comprises controllable dividing means M_{D} arranged downstream of U_{M}, wherein the process according to (iii) further comprises
   (iii.3) passing the stream S_{M} into the dividing means M_{D}, obtaining a stream S′_{M} being x volume-% of S_{M}, with 0≤x≤100, and obtaining a stream S″_{M} being (100-x) volume-% of S_{M}.
13. The method of embodiment 12, wherein the process stage (iii) further comprises
   (iii.3) passing the stream S_{M} into the dividing means M_{D}, obtaining a stream S′_{M} being x volume-% of S_{M}, with 0<x≤100, and obtaining a stream S″_{M} being (100-x) volume-% of S_{M};
   (iii.4) passing the stream S′m as feed stream into U_{M}.
14. The method of any one of embodiments 1 to 13, wherein from 60 to 100 volume-%, more preferably from 70 to 100 volume-%, more preferably from 80 to 100 volume-%, more preferably from 90 to 100 volume-%, more preferably from 95 to 100 volume-%, more preferably from 99 to 100 volume-% of the H₂-rich stream S_{H} consist of H₂.
15. The method of any one of embodiments 1 to 14, wherein for ΔE>0 and ΔH≤0, the process stage (iv′) comprises
   (iv′) passing the stream S_{H} obtained from U_{M} through M_{E}, obtaining a stream S′_{H} being y volume-% of S_{H}, with 0<y<100, and obtaining a stream S″_{H} being (100-y) volume-% of S_{H};
   wherein the stream S″_{H} or a part thereof is preferably put to further use, wherein said further use preferably comprises the use of S″_{H} as a heat source in one or more units other than U_{C}.
16. The method of any one of embodiments 1 to 15, wherein for ΔE≤0, the process stage (v″) comprises putting the stream S″_{H} or a part thereof to further use, wherein said further use preferably comprises the use of S″_{H} as a heat source in one or more units other than U_{C}.
17. The method of any one of embodiments 1 to 16, being at least partially computer-implemented.
18. The method of embodiment 17, wherein at least one of E_{UC}, E_{EXT-S} and ΔE is determined by a computer-supported control system, preferably wherein at least two of E_{UC}, E_{EXT-S} and ΔE are determined by a computer-supported control system, more preferably wherein all three of E_{UC}, E_{EXT-S} and ΔE are determined by a computer-supported control system.
19. The method of embodiment 17 or 18, wherein at least one M_{F}, U_{C}, M_{E} and U_{M} is determined by a computer-supported control system, preferably wherein at least two of M_{F}, U_{C}, M_{E} and U_{M} are determined by a computer-supported control system, more preferably wherein at least three of M_{F}, U_{C}, M_{E} and U_{M} are determined by a computer-supported control system, more preferably wherein all of M_{F}, U_{C}, M_{E} and U_{M} are determined by a computer-supported control system.
20. An integrated production plant, comprising
   (1) supply means M_{F} for providing one or more hydrocarbon feed streams S_{F} to a unit U_{C} for heat-consuming hydrocarbon conversion, wherein the supply means M_{F} are connected to U_{C} for passing at least one of said hydrocarbon feed stream S_{F} into U_{C} to obtain from U_{C} one or more product streams S and a light hydrocarbon conversion off-gas stream S_{O} comprising CH₄;
   (2) the unit U_{C} which comprises heating means M_{H} for providing heat to the hydrocarbon conversion, wherein said heating means M_{H} comprise means for generating heat from one or more heat sources H_{S} and wherein said one or more heat sources H_{S} comprise H₂;
   (3) controllable supply means M_{E} for providing one or more plant-external sustainable heat sources H_{EXT-S}, one or more plant-external fossil heat sources H_{EXT-F}, and one or more plant-internal heat sources H_{INT}, said plant-internal heat sources H_{INT} comprising H₂, wherein the supply means M_{E} are connected to U_{C} for passing at least one of said heat sources H_{EXT-S}, H_{EXT-F} and H_{INT} to the heating means M_{H};
   (4) a methane pyrolysis unit U_{M} for subjecting the light hydrocarbon conversion off-gas stream S_{O} to methane pyrolysis to obtain a hydrogen-rich gas stream S_{H} and a carbon stream S_{K};
   (5) optionally a gas separation unit U_{GS} comprised in U_{M} for separating a gas stream S_{M} obtained from methane pyrolysis into the stream S_{H} and a stream S_{M} being enriched in methane compared to S_{HM};
   (6) and optionally controllable dividing means M_{D} for optionally dividing the stream S_{M} into a part stream S′_{M} and a stream S″_{M}, wherein M_{D} is connected to U_{M} for passing S_{M} into M_{D} and passing S′_{M} into U_{M}.
21. The integrated production plant of embodiment 20, wherein the unit U_{C} for heat-consuming hydrocarbon conversion comprises a cracking unit U_{CC}, preferably a thermal cracking unit, more preferably a steam cracking unit.
22. The integrated production plant of embodiment 21, wherein U_{C} further comprises a separation unit U_{S} for treating a cracked gas stream S_{C}, wherein U_{S} comprises at least one demethanizing unit, wherein from said demethanizing unit the stream S_{O} is obtained and wherein from U_{S}, the one or more product streams are obtained.
23. The integrated production plant of any one of embodiments 20 to 22, further comprising a computer-supported control system for controlling at least one of M_{F}, U_{C}, M_{H}, M_{E}, U_{M}, U_{GS} and M_{D}.
24. A computer program comprising instructions which, when the program is executed by the computer-supported control system as defined in embodiment 23, cause the system to perform the method of any one of embodiments 1 to 19.
25. A non-transient computer-readable medium containing instructions which, when executed by one or more processors, cause the one or more processors to perform the program according to embodiment 24.
26. Use of the integrated production plant according to any one of embodiments 20 to 23 for carrying out the method according to any one of embodiments 1 to 19.
27. A method of using the integrated production plant according to any one of embodiments 20 to 23 for carrying out the method according to any one of embodiments 1 to 19.
28. A process for carrying out a heat-consuming hydrocarbon conversion in an integrated production plant which comprises
   (1) supply means M_{F} for providing one or more hydrocarbon feed streams S_{F} to a unit U_{C} for heat-consuming hydrocarbon conversion, wherein the supply means M_{F} are connected to U_{C} for passing at least one of said hydrocarbon feed stream S_{F} into U_{C} to obtain from U_{C} one or more product streams S and a light hydrocarbon conversion off-gas stream S_{O} comprising CH₄;
   (2) the unit U_{C} which comprises heating means M_{H} for providing heat to the hydrocarbon conversion, wherein said heating means M_{H} comprise means for generating heat from one or more heat sources H_{S} and wherein said one or more heat sources H_{S} comprise H₂;
   (3) controllable supply means M_{E} for providing one or more plant-external sustainable heat sources H_{EXT-S}, one or more plant-external fossil heat sources H_{EXT-F}, and one or more plant-internal heat sources H_{INT}, said plant-internal heat sources H_{INT} comprising H₂, wherein the supply means M_{E} are connected to U_{C} for passing at least one of said heat sources H_{EXT-S}, H_{EXT-F} and H_{INT} to the heating means M_{H};
   (4) a methane pyrolysis unit U_{M} for subjecting the light hydrocarbon conversion off-gas stream S_{O} to methane pyrolysis to obtain a hydrogen-rich gas stream S_{H} and a carbon stream S_{K};
   (5) optionally a gas separation unit U_{GS} comprised in U_{M} for separating a gas stream S_{M} obtained from methane pyrolysis into the stream S_{H} and a stream S_{M} being enriched in methane compared to S_{HM};
   (6) and optionally controllable dividing means M_{D} for optionally dividing the stream S_{M} into a part stream S′_{M} and a stream S″_{M}, wherein M_{D} is connected to U_{M} for passing S_{M} into M_{D} and passing S′m into U_{M};
   wherein the process comprises
   (i) passing one or more heat sources according to (3) from M_{E} to My in an amount sufficient for generating heat in M_{H} meeting the heat demand E_{UC} of U_{C};
   (ii) passing at least one hydrocarbon feed stream S_{F} into U_{C} and subjecting the at least one feed stream S_{F} to hydrocarbon conversion in U_{C}, obtaining the one or more product streams S and S_{O};
   (iii) passing S_{O} into U_{M} and subjecting S_{O} to methane pyrolysis in U_{M}, obtaining S_{H} and S_{K};
   wherein during the process, parameters E_{UC}, E_{EXT-S}, ΔE, H_{2N}, H_{2P} and ΔH as defined in any one of embodiments 1 to 16 are determined,
   wherein for ΔE>0 and ΔH>0, the process further comprises
   (iv) passing the stream S_{H} obtained from U_{M} through M_{E}, obtaining a stream S′_{H} being y volume-% of S_{H}, with y=100;
   (v) passing the stream S′_{H} obtained from M_{E} as H_{INT} into M_{H};
   (vi) passing at least one of the one or more plant-external fossil heat sources H_{EXT-F} via M_{E} into My in an amount sufficient to generate heat in an amount (ΔE−E_{H2P}) in M_{H};
   wherein for ΔE>0 and ΔH≤0, the process further comprises
   (iv′) passing the stream S_{H} obtained from U_{M} through M_{E}, obtaining a stream S′_{H} being y volume-% of S_{H}, with 0<y≤100;
   (v′) passing the stream S′_{H} obtained from M_{E} as H_{INT} into M_{H};
   wherein for ΔE≤0, the process further comprises
   (iv″) passing the stream S_{H} through M_{E}, obtaining a stream S″_{H} being 100 volume-% of S_{H};
   (v″) removing the stream S″_{H} from M_{E}.
29. The process of embodiment 28, wherein U_{C} further comprises a separation unit U_{S}, wherein according to (ii), the process comprises
   (ii.1) passing at least one hydrocarbon feed stream S_{F} into U_{C} and subjecting the at least one feed stream S_{F} to hydrocarbon conversion in U_{C}, obtaining a cracked gas stream S_{C};
   (ii.2) passing the cracked gas stream S_{C} obtained according to (ii.1) to a separation unit U_{S} comprising at least one demethanizing unit, obtaining from said demethanizing unit the stream S_{O}, wherein from U_{S}, the one or more product streams S are obtained.
30. The process of embodiment 28 or 29, wherein the methane pyrolysis unit U_{M} according to (4) comprises a gas separation unit U_{GS}, wherein the process according to (iii) comprises
   (iii.1) passing S_{O} into U_{M} and subjecting S_{O} to methane pyrolysis in U_{M}, obtaining a stream S_{HM} comprising methane and hydrogen, and obtaining the stream S_{K};
   (iii.2) passing the stream S_{HM} into U_{GS}, obtaining a stream S_{M} being enriched in methane compared to S_{HM}, and obtaining the stream S_{H}.
31. The process of embodiment 30, wherein the integrated plant further comprises controllable dividing means M_{D} arranged downstream of U_{M}, wherein the process according to (iii) further comprises
   (iii.3) passing the stream S_{M} into the dividing means M_{D}, obtaining a stream S′_{M} being x volume-% of S_{M}, with 0≤x≤100, and obtaining a stream S″_{M} being (100-x) volume-% of S_{M}.
32. The process of embodiment 31, according to (iii) further comprising
   (iii.3) passing the stream S_{M} into the dividing means M_{D}, obtaining a stream S′_{M} being x volume-% of S_{M}, with 0<x≤100, and obtaining a stream S′_{M} being (100-x) volume-% of S_{M}.
   (iii.4) passing the stream S′_{M} as feed stream into U_{M}.
33. The process of any one of embodiments 28 to 32, according to (iv′) comprising
   (iv′) passing the stream S_{H} obtained from U_{M} through M_{E}, obtaining a stream S′_{H} being y volume-% of S_{H}, with 0<y<100, and obtaining a stream S″_{H} being (100-y) volume-% of S_{H};
   wherein the stream S″_{H} or a part thereof is preferably put to further use, wherein said further use preferably comprises the use of S″_{H} as a heat source in one or more units other than U_{C}.
34. The process of any one of embodiments 28 to 33, according to (v″) comprising putting the stream S″_{H} or a part thereof to further use, wherein said further use preferably comprises the use of S″_{H} as a heat source in one or more units other than U_{C}.
35. The method of any one of embodiments 28 to 34, being at least partially computer-implemented.

### DESCRIPTION OF THE FIGURES

FIG. 1 is a schematic representation of interconnected units and means of an integrated production plant of the present invention and used for carrying out the method of the present invention, and of streams passed into and obtained from said units and means. In this figure, supply means M_{F} are shown for for providing one or more hydrocarbon feed streams S_{F} to a unit U_{C} for heat-consuming hydrocarbon conversion. In this unit U_{C}, the hydrocarbon conversion take place, and from U_{C}, the one or more product streams S and the light hydrocarbon conversion off-gas stream S_{O} comprising CH₄ are obtained. Further, the unit U_{C} comprises heating means M_{H} which in turn comprise means for generating heat from one or more heat sources H_{S} (plant-external sustainable heat sources H_{EXT-S} plant-external fossil heat sources H_{EXT-S}, and plant-internal heat sources H_{INT}) wherein said one or more heat sources H_{S} comprise hydrogen (H₂). The light hydrocarbon conversion off-gas stream S_{O} comprising CH₄ is then passed to the methane pyrolysis unit U_{M} from which the H₂-rich stream S_{H} and the carbon stream S_{K} are obtained.

If, according to the control method of the present invention, the determined value ΔE=E_{UC}−E_{EXT-S}, i.e. the difference of the heat demand of U_{C} and the heat which can be supplied to U_{C} via the one or more plant-external sustainable heat sources H_{EXT-S}, indicated by the dotted arrow labelled “ext”, is greater than zero, i.e. the heat demand of U_{C} cannot be met solely by the plant-external sustainable heat sources, the following parameters are determined:
the amount of H₂ which is necessary to produce ΔE in the means M_{H}. This amount is referred to herein as H₂N;
the maximum amount of H₂ which can be produced in U_{M}, which maximum amount of H₂ is then contained in the stream S_{H}. This amount is referred to herein as H_{2P} and based on H_{2P}, an amount of heat can be produced which amount is referred to herein as E_{H2P};
the parameter ΔH which is defined as the difference H_{2N}−H_{2P}.

If ΔH is greater than zero, which means that the heat demand ΔE cannot be covered by the maximum amount H_{2P} alone, the supply means M_{E} are controlled so that the maximum amount H_{2P} is passed via into M_{H} via the H₂-rich stream S_{H} and S′_{H} so as to produce E_{H2P} in M_{H}. As far as the remaining amount of heat is concerned in this case, at least one of the plant-external fossil heat sources H_{EXT-F} is passed via M_{E} into M_{H} in an amount which is sufficient to generate said remaining amount of heat (ΔE−E_{H2P}).

If ΔH is less than or equal to zero, which means that the heat demand ΔE can be covered by the amount of H₂ which is available via U_{M}, the supply means M_{E} are controlled so that an amount of H₂ is passed into M_{H} via the H₂-rich stream S_{H} and S′_{H} which is sufficient to produce said amount of ΔE in M_{H}. The supply means M_{E} can be controlled in a manner so that only a suitable part of S_{H}, namely S′_{H}, sufficient for producing ΔE, is passed into M_{H} and a part of S_{H}, S″_{H}, is put to one or more further suitable uses.

If, according to the control method of the present invention, the determined value ΔE=E_{UC}−E_{EXT-S}, i.e. the difference of the heat demand of U_{C} and the heat which can be supplied to U_{C} via the one or more plant-external sustainable heat sources H_{EXT-S}, is less than or equal to zero, i.e. the heat demand of U_{C} can be met solely by the plant-external sustainable heat sources H_{EXT-S}, the supply means M_{E} are controlled in such a manner that no H₂ is passed into M_{H} via the H₂-rich stream S_{H}. In this case, the supply means M_{E} are controlled in a manner so that no part of S_{H} is passed into M_{H} and S″_{H}, which is identical to S_{H}, is put to one or more further suitable uses.

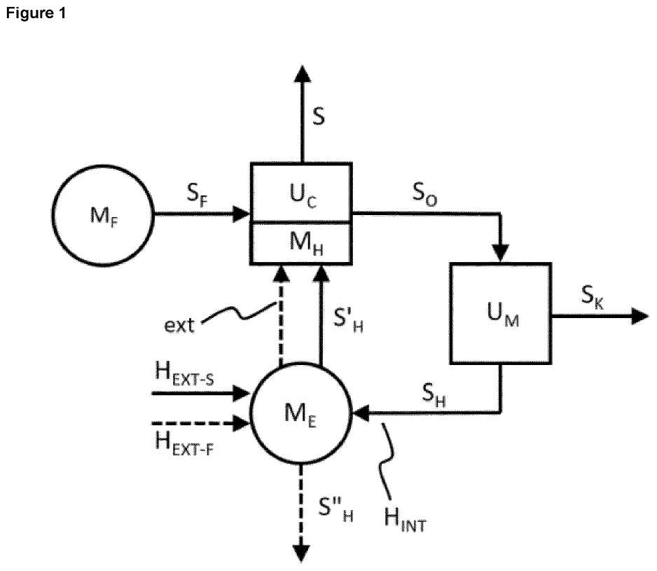

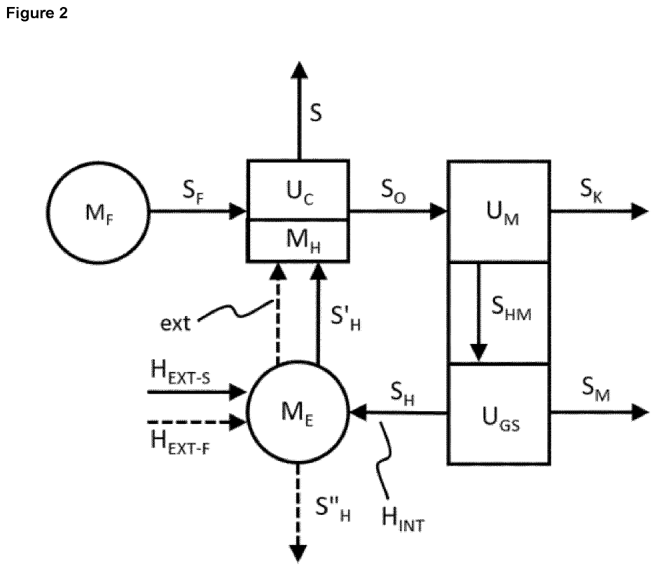

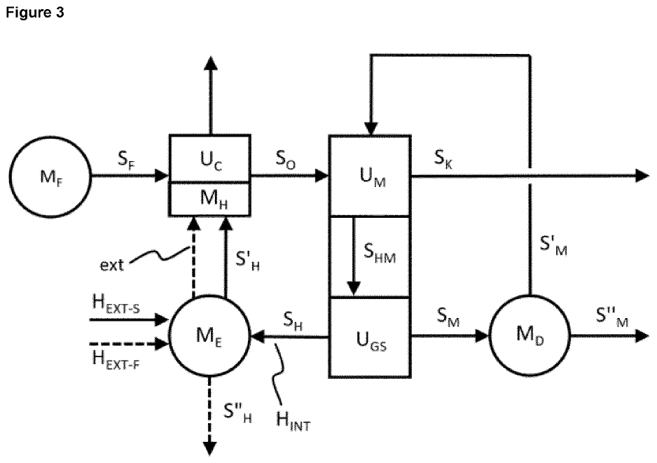

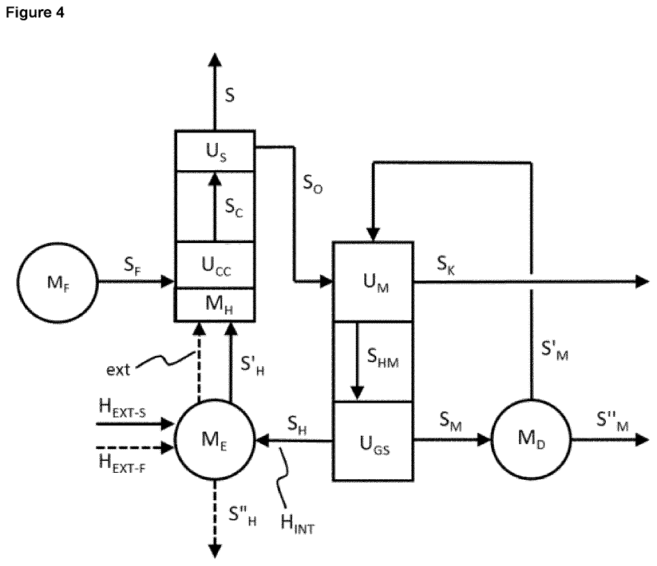

FIG. 2 is a schematic representation of interconnected units and means of an integrated production plant of the present invention and used for carrying out the method of the present invention, and of streams passed into and obtained from said units and means. Compared with FIG. 1, the methane pyrolysis unit U_{M} comprises a gas separation unit U_{GS}. According to this embodiment, the gas stream S_{HM} which is obtained from methane pyrolysis and which mainly consists of hydrogen and methane is passed into U_{GS} from which the hydrogen-rich stream S_{H} is obtained which is enriched in H₂ compared to S_{HM}. Further from U_{GS}, a CH₄-rich stream S_{M} is obtained which is enriched in CH₄ compared to S_{HM}.

FIG. 3 is a schematic representation of interconnected units and means of an integrated production plant of the present invention and used for carrying out the method of the present invention, and of streams passed into and obtained from said units and means. Compared to FIG. 2, a configuration is shown which additionally comprises controllable dividing means M_{D}. According to this embodiment, the CH₄-rich stream can be suitably divided into two streams, S′_{M} and S″_{M}, which preferably have the same composition as S_{M}. As shown, the part stream S′_{M} is then passed (back) as feed stream to methane pyrolysis in U_{M}. Thus, the controllable means M_{D} can be used for recycling at least a part of the methane which is not converted during methane pyrolysis and comprised in S_{HM} to methane pyrolysis.

FIG. 4 is a schematic representation of interconnected units and means of an integrated production plant of the present invention and used for carrying out the method of the present invention, and of streams passed into and obtained from said units and means. Compared to FIG. 3, a preferred configuration of the unit U_{C} is shown, wherein U_{C} comprises a cracking unit U_{CC}, preferably a thermal cracking unit, more preferably a steam cracking unit. Further, the unit U_{C} comprises a separation unit U_{S} which is used for a downstream treatment of the cracked gas stream S_{C} obtained from U_{CC}. This separation unit U_{S} comprises a demethanizing stage (not shown) from which the light hydrocarbon conversion off-gas stream S_{O} is obtained.

1.-17. (canceled)

18. A method for controlling a process carried out in an integrated production plant, wherein the integrated production plant comprises:
(1) supply means M_{F} for providing one or more hydrocarbon feed streams S_{F} to a unit U_{C} for heat-consuming hydrocarbon conversion to obtain from U_{C} one or more product streams S and a light hydrocarbon conversion off-gas stream S_{O} comprising CH₄;
(2) the unit U_{C} which comprises heating means M_{H} for providing heat to the hydrocarbon conversion, wherein said heating means M_{H} comprise means for generating heat from one or more heat sources H_{S} and wherein said one or more heat sources H_{S} comprise H₂;
(3) controllable supply means M_{E} for providing one or more plant-external sustainable heat sources H_{EXT-S}, one or more plant-external fossil heat sources H_{EXT-F}, and one or more plant-internal heat sources H_{INT}, said plant-internal heat sources H_{INT} comprising H₂, for passing at least one of said heat sources H_{EXT-S}, H_{EXT-F} and H_{INT} to the heating means M_{H}; and
(4) a methane pyrolysis unit U_{M} for subjecting the light hydrocarbon conversion off-gas stream S_{O} to methane pyrolysis to obtain a hydrogen-rich gas stream S_{H} and a carbon stream S_{K};
wherein the process comprises:
(i) passing one or more heat sources according to (3) from M_{E} to M_{H} in an amount sufficient for generating heat in M_{H} meeting the heat demand E_{UC} of U_{C};
(ii) passing at least one hydrocarbon feed stream S_{F} into U_{C} and subjecting the at least one feed stream S_{F} to hydrocarbon conversion in U_{C}, obtaining the one or more product streams S and S_{O}; and
(iii) passing S_{O} into U_{M} and subjecting S_{O} to methane pyrolysis in U_{M}, obtaining S_{H} and S_{K};
wherein the method for controlling the process comprises:
(a) determining the heat demand E_{UC} of the hydrocarbon conversion in U_{C} to be provided by M_{H};
(b) determining the amount E_{EXT-S} of heat available for U_{C} from the one or more plant-external sustainable heat sources H_{EXT-S}; and

| (c.1) | if ΔE > 0, | |
|---|---|---|
| | (c.1.1) | determining H_{2N}, the amount of H₂ necessary to produce ΔE in M_{H}; |
| | (c.1.2) | determining H_{2P}, the maximum amount of H₂, producible in U_{M} to be |
| | | contained in S_{H}, to produce an amount E_{H2P} of heat in M_{H}; |
| | (c.1.3) | determining ΔH = H_{2N} − H_{2P}; and |
| | |
|---|---|
| (c.1.3.1) | if ΔH > 0, |
| | controlling the supply means M_{E} so that H_{2P} is passed into |
| | M_{H} via S_{H}; |
| | wherein the process further comprises: |
| | |
|---|---|
| (iv) | passing the stream S_{H} obtained from U_{M} through |
| | M_{E}, obtaining a stream S′_{H} being y volume-% of |
| | S_{H}, with y = 100; |
| (v) | passing the stream S′_{H} obtained from M_{E} as H_{INT} |
| | into M_{H}; and |
| (vi) | passing at least one of the one or more plant- |
| | external fossil heat sources H_{EXT-F} via M_{E} into M_{H} |
| | in an amount sufficient to generate heat in an |
| | amount (ΔE − E_{H2P}) in M_{H}; |
| | |
|---|---|
| (c.1.3.2) | if ΔH ≤ 0, |
| | controlling the supply means M_{E} so that an amount of H₂ |
| | is passed into M_{H} via S_{H} which is sufficient to produce |
| | ΔE in M_{H}; |
| | wherein the process further comprises: |
| | |
|---|---|
| (iv′) | passing the stream S_{H} obtained from U_{M} through |
| | M_{E}, obtaining a stream S′_{H} being y volume-% of |
| | S_{H}, with 0 < y ≤ 100; |
| (v′) | passing the stream S′_{H} obtained from M_{E} as H_{INT} |
| | into M_{H}; and |
| (c.2) | if ΔE ≤ 0, | |
|---|---|---|
| | controlling the suply means M_{E} so that no H₂ is passed into M_{H} via S_{H}; | |
| | wherein the process further comprises: | |
| | (iv″) | passing the stream S_{H} through M_{E}, obtaining a stream S″_{H} being 100 |
| | | volume-% of S_{H}; and |
| | (v″) | removing the stream S″_{H} from M_{E}. |

controlling the supply means M_{E} so that no H₂ is passed into M_{H} via S_{H};

19. The method of claim 18, wherein from 40 to 100 volume-% of S_{O} consist of CH₄.

20. The method of claim 18, wherein the unit U_{C} for heat-consuming hydrocarbon conversion comprises a cracking unit U_{CC}.

21. The method of claim 20, wherein U_{C} further comprises a separation unit U_{S}, wherein according to (ii), the process comprises:
(ii.1) passing at least one hydrocarbon feed stream S_{F} into U_{C} and subjecting the at least one feed stream S_{F} to hydrocarbon conversion in U_{C}, obtaining a cracked gas stream S_{C}; and
(ii.2) passing the cracked gas stream S_{C} obtained according to (ii.1) to a separation unit Us comprising at least one demethanizing unit, obtaining from said demethanizing unit the stream S_{O}, wherein from U_{S}, the one or more product streams S are obtained.

22. The method of claim 18 wherein from 96 to 100 volume-% of S_{O} consists of CH₄; wherein from 0 to 4 volume-% of S_{O} consist of H₂.

23. The method of claim 18, wherein the one or more hydrocarbon feed streams S_{F} comprise at least one of a hydrocarbon feed stream comprising fossil hydrocarbons; a hydrocarbon feed stream comprising recycled hydrocarbons; and a hydrocarbon feed stream comprising biohydrocarbons.

24. The method of claim 18, wherein the one or more plant-external sustainable heat sources H_{EXT-S} according to (3) comprise one or more of solar energy, wind energy, hydro energy, tidal energy, geothermal energy, and biomass energy, ammonia obtained from renewable sources, biomethane, bio-LNG and H₂ obtained from renewable sources.

25. The method of claim 18, wherein the carbon comprised in S_{K} is stored or put to further use for producing of one or more of aluminium and steel, for preparing printing inks, for preparing electrodes, for polymer blending, as additive for construction materials, or for soil improvement.

26. The method of claim 18, wherein the methane pyrolysis unit U_{M} according to (4) comprises a gas separation unit U_{GS}, wherein the process according to (iii) comprises:
(iii.1) passing S_{O} into U_{M} and subjecting S_{O} to methane pyrolysis in U_{M}, obtaining a stream S_{HM} comprising methane and hydrogen, and obtaining the stream S_{K}; and
(iii.2) passing the stream S_{HM} into U_{GS}, obtaining a stream S_{M} being enriched in methane compared to S_{HM}, and obtaining the stream S_{H}.

27. The method of claim 26, wherein the integrated plant further comprises controllable dividing means M_{D} arranged downstream of U_{M}, wherein the process according to (iii) further comprises:
(iii.3) passing the S_{M} the stream into the dividing means M_{D}, obtaining a stream S′_{M} being x volume-% of S_{M}, with 0≤x≤100, and obtaining a stream S″_{M} being (100-x) volume-% of S_{M}.

28. The method of claim 27, wherein the process stage (iii) further comprises:
(iii.3) passing the S_{M} the stream into the dividing means M_{D}, obtaining a stream S′_{M} being x volume-% of S_{M}, with 0<x≤100, and obtaining a stream S″_{M} being (100-x) volume-% of S_{M}; and
(iii.4) passing the stream S′_{M} as feed stream into U_{M}.

29. The method of claim 18, wherein from 60 to 100 volume-% of the H₂-rich stream S_{H} consist of H₂.

30. The method of claim 18, wherein for ΔE>0 and ΔH≤0, the process stage (iv′) comprises:
(iv′) passing the stream S_{H} obtained from U_{M} through M_{E}, obtaining a stream S′_{H} being y volume-% of S_{H}, with 0<y<100, and obtaining a stream S″_{H} being (100-y) volume-% of S_{H};
wherein the stream S″_{H} or a part thereof is put to further use, wherein said further use
comprises the use of S″_{H} as a heat source in one or more units other than U_{C}.

31. The method of claim 18, wherein for ΔE≤0, the process stage (v″) comprises putting the stream S″_{H} or a part thereof to further use, wherein said further use comprises the use of S″_{H} as a heat source in one or more units other than U_{C}.

32. An integrated production plant for carrying out the method according to claim 18, comprising:
(1) supply means M_{F} for providing one or more hydrocarbon feed streams S_{F} to a unit U_{C} for heat-consuming hydrocarbon conversion, wherein the supply means M_{F} are connected to U_{C} for passing at least one of said hydrocarbon feed stream S_{F} into U_{C} to obtain from U_{C} one or more product streams S and a light hydrocarbon conversion off-gas stream S_{O} comprising CH₄;
(2) the unit U_{C} which comprises heating means M_{H} for providing heat to the hydrocarbon conversion, wherein said heating means M_{H} comprise means for generating heat from one or more heat sources H_{S} and wherein said one or more heat sources H_{S} comprise H₂;
(3) controllable supply means M_{E} for providing one or more plant-external sustainable heat sources H_{EXT-S}, one or more plant-external fossil heat sources H_{EXT-F}, and one or more plant-internal heat sources H_{INT}, said plant-internal heat sources H_{INT} comprising H₂, wherein the supply means M_{E} are connected to U_{C} for passing at least one of said heat sources H_{EXT-S}, H_{EXT-F} and H_{INT} to the heating means M_{H};
(4) a methane pyrolysis unit U_{M} for subjecting the light hydrocarbon conversion off-gas stream S_{O} to methane pyrolysis to obtain a hydrogen-rich gas stream S_{H} and a carbon stream S_{K};
(5) optionally a gas separation unit U_{GS} comprised in U_{M} for separating a gas stream S_{M} obtained from methane pyrolysis into the stream S_{H} and a stream S_{M} being enriched in methane compared to S_{HM}; and
(6) and optionally controllable dividing means M_{D} for optionally dividing the stream S_{M} into a part stream S′_{M} and a stream S″_{M}, wherein M_{D} is connected to U_{M} for passing S_{M} into M_{D} and passing S′_{M} into U_{M};
wherein the unit U_{C} for heat-consuming hydrocarbon conversion comprises a cracking unit U_{CC}; and
wherein U_{C} further comprises a separation unit U_{S} for treating a cracked gas stream S_{C}, wherein U_{S} comprises at least one demethanizing unit, wherein from said demethanizing unit the stream S_{O} is obtained and wherein from U_{S}, the one or more product streams are obtained.

33. The integrated production plant of claim 32, further comprising a computer-supported control system for controlling at least one of M_{F}, U_{C}, M_{H}, M_{E}, U_{M}, U_{GS} and M_{D}.

34. A computer program comprising instructions which, when the program is executed by the computer-supported control system as defined in claim 33, cause the system to perform a method of for controlling a process carried out in an integrated production plant, wherein the integrated production plant comprises:
(5) supply means M_{F} for providing one or more hydrocarbon feed streams S_{F} to a unit U_{C} for heat-consuming hydrocarbon conversion to obtain from U_{C} one or more product streams S and a light hydrocarbon conversion off-gas stream S_{O} comprising CH₄;
(6) the unit U_{C} which comprises heating means M_{H} for providing heat to the hydrocarbon conversion, wherein said heating means M_{H} comprise means for generating heat from one or more heat sources H_{S} and wherein said one or more heat sources H_{S} comprise H₂;
(7) controllable supply means M_{E} for providing one or more plant-external sustainable heat sources H_{EXT-S}, one or more plant-external fossil heat sources H_{EXT-F}, and one or more plant-internal heat sources H_{INT}, said plant-internal heat sources H_{INT} comprising H₂, for passing at least one of said heat sources H_{EXT-S}, H_{EXT-F} and H_{INT} to the heating means M_{H}; and
(8) a methane pyrolysis unit U_{M} for subjecting the light hydrocarbon conversion off-gas stream S_{O} to methane pyrolysis to obtain a hydrogen-rich gas stream S_{H} and a carbon stream S_{K};
wherein the process comprises:
(vii) passing one or more heat sources according to (3) from M_{E} to M_{H} in an amount sufficient for generating heat in M_{H} meeting the heat demand E_{UC} of U_{C};
(viii) passing at least one hydrocarbon feed stream S_{F} into U_{C} and subjecting the at least one feed stream S_{F} to hydrocarbon conversion in U_{C}, obtaining the one or more product streams S and S_{O}; and
(ix) passing S_{O} into U_{M} and subjecting S_{O} to methane pyrolysis in U_{M}, obtaining S_{H} and S_{K};
wherein the method for controlling the process comprises:
(d) determining the heat demand E_{UC} of the hydrocarbon conversion in U_{C} to be provided by M_{H};
(e) determining the amount E_{EXT-S} of heat available for U_{C} from the one or more plant-external sustainable heat sources H_{EXT-S}; and
(f) determining ΔE=E_{UC}−E_{EXT-S} and

| (c.1) | if ΔE > 0, | |
|---|---|---|
| | (c.1.1) | determining H_{2N}, the amount of H₂ necessary to produce ΔE in M_{H}; |
| | (c.1.2) | determining H_{2P}, the maximum amount of H₂, producible in U_{M} to be |
| | | contained in S_{H}, to produce an amount E_{H2P} of heat in M_{H}; |
| | (c.1.3) | determining ΔH = H_{2N} − H_{2P}; and |
| | |
|---|---|
| (c.1.3.1) | if ΔH > 0, |
| | controlling the supply means M_{E} so that H_{2P} is passed into |
| | M_{H} via S_{H}; |
| | wherein the process further comprises: |
| | |
|---|---|
| (x) | passing the stream S_{H} obtained from U_{M} through |
| | M_{E}, obtaining a stream S′_{H} being y volume-% of |
| | S_{H}, with y = 100; |
| (xi) | passing the stream S′_{H} obtained from M_{E} as H_{INT} |
| | into M_{H}; and |
| (xii) | passing at least one of the one or more plant- |
| | external fossil heat sources H_{EXT-F} via M_{E} into M_{H} |
| | in an amount sufficient to generate heat in an |
| | amount (ΔE − E_{H2P}) in M_{H}; |
| | |
|---|---|
| (c.1.3.2) | if ΔH ≤ 0, |
| | controlling the supply means M_{E} so that an amount of H₂ |
| | is passed into M_{H} via S_{H} which is sufficient to produce |
| | ΔE in M_{H}; |
| | wherein the process further comprises: |
| | |
|---|---|
| (iv′) | passing the stream S_{H} obtained from U_{M} through |
| | M_{E}, obtaining a stream S′_{H} being y volume-% of |
| | S_{H}, with 0 < y ≤ 100; |
| (v′) | passing the stream S′_{H} obtained from M_{E} as H_{INT} |
| | into M_{H}; and |
| (c.2) | if ΔE ≤ 0, | |
|---|---|---|
| | controlling the supply means M_{E} so that no H₂ is passed into M_{H} via S_{H}; | |
| | wherein the process further comprises: | |
| | (iv″) | passing the stream S_{H} through M_{E}, obtaining a stream S″_{H} being 100 |
| | | volume-% of S_{H}; and |
| | (v″) | removing the stream S″_{H} from M_{E}. |
